SEQUENCE LISTING (6 sequences)

SEQ ID NO: 1 - protein, 505 aa, Equus caballus papillomavirus type 1
MASYWSSNSQKVYLPPTTLTKAVSTDTYVTRLGIYYHGHSDRLLTVGHPFYEITNGRDQTMRVPKVSANQFRVFRVILPNPNKFALPDSNVFDPDSERLVWAVKAMEICRGQPIGPQVTGHPLFNRFEDVENPAVYKPGFGTGDKRQNMASDYKQIQMVVLGCRPALGEHWGKTRSICPGIQNNVLTGDCPAIELFHTTIEDGDMVDIGLGNLDFAQLQADKSGAPLDIVQSICKYPDTLKMAQEITGDTMFFSARREQSYLRHMMTRAGINKEAIPEALYIKGATEPQNTVGTSVYCGVVSGSLFSSDAQIFNRPFWLNQAQGLNNGIAWNNQLFVTAVDNTRATNFTITVATDEREKDTYDAGSFNAYLRHVESYELQFVFELCKVKLTPENLTILHQQDPGILKGWELGVTPPSGSVLEDTYRYINSVATKCPPNPPEEVQEDPWGRFAFWRVDLSERFSLDLDQFPLGRRFLALSAPRTRTSAAKRKTPVSAKSSKQRRKG

SEQ ID NO: 2 - DNA, 1518 bp, Equus caballus papillomavirus type 1
atggcgtcctactggtcctccaactcacagaaggtgtacctgcccccaaccacattgactaaggccgtatccacggacacctatgtgaccagattgggtatatactatcatggtcacagtgaccggcttctgacggtgggccacccattctacgagataacaaatgggcgtgaccaaaccatgcgggtgcccaaggtgtctgcaaatcagtttcgggttttcagagtgattcttccaaaccccaacaaatttgctcttcctgatagcaatgtttttgatccagattcagaaaggctggtgtgggctgtcaaggctatggagatttgcagaggtcagcccatagggcctcaggtgactgggcacccattgtttaataggtttgaagatgtggaaaaccctgcagtttataagccagggttcggcacgggtgacaagagacagaatatggcatcagactataagcaaatacagatggtggtactgggctgcaggcctgccttgggggagcactggggtaagacacgcagcatttgcccaggcattcaaaataatgttctcaccggtgactgccctgctatagagctgtttcacaccactatagaggatggagatatggtagacataggtctagggaatctggactttgcacagctgcaggccgataagtcaggtgcccccctggatatagttcagtcaatttgcaaatatccggatacattgaaaatggcccaggagattactggtgacaccatgttttttagtgctaggcgggagcagagctatcttagacacatgatgacacgtgccggtatcaacaaagaggctataccagaggccctatatataaagggcgccacagagccacagaatactgtgggcacctctgtttactgtggggtggtgtctggctccttatttagtagcgatgcacagatttttaacaggccattctggctaaatcaggcccagggtctaaataatggcatagcctggaataatcagctttttgtcacggcggtggataatacccgggccactaatttcactataaccgtggcgacagatgagagggagaaggatacctatgatgctggaagctttaatgcttacctgagacatgtagaatcctatgagctgcagtttgtatttgaactttgtaaggtcaagctgacccctgagaacctgacaatcctgcaccagcaggaccccggcatacttaagggctgggagctgggggtgacccccccttcgggttcggtcttggaggacacctaccgctacattaattctgtagcgaccaagtgcccgcctaatccacctgaggaggtgcaggaggatccctggggacggttcgcattttggagagttgacctttcagagcgcttttctcttgaccttgaccagtttccattgggaagaaggtttttggccctttccgcaccccgtacccgcacgtccgcagctaagcgtaagaccccggtttctgccaagtcttctaaacaaagaagaaagggctaa

SEQ ID NO: 3 - protein, 495 aa, Bovine papillomavirus type 1
MALWQQGQKLYLPPTPVSKVLCSETYVQRKSIFYHAETERLLTIGHPYYPVSIGAKTVPKVSANQYRVFKIQLPDPNQFALPDRTVHNPSKERLVWAVIGVQVSRGQPLGGTVTGHPTFNALLDAENVNRKVTTQTTDDRKQTGLDAKQQQILLLGCTPAEGEYWTTARPCVTDRLENGACPPLELKNKHIEDGDMMEIGFGAANFKEINASKSDLPLDIQNEICLYPDYLKMAEDAAGNSMFFFARKEQVYVRHIWTRGGSEKEAPTTDFYLKNNKGDATLKIPSVHFGSPSGSLVSTDNQIFNRPYWLFRAQGMNNGIAWNNLLFLTVGDNTRGTNLTISVASDGTPLTEYDSSKFNVYHRHMEEYKLAFILELCSVEITAQTVSHLQGLMPSVLENWEIGVQPPTSSILEDTYRYIESPATKCASNVIPAKEDPYAGFKFWNIDLKEKLSLDLDQFPLGRRFLAQQGAGCSTVRKRRISQKTSSKPAKKKKK

SEQ ID NO: 4 - DNA, 1488 bp, Bovine papillomavirus type 1
atggcgttgtggcaacaaggccagaagctgtatctccctccaacccctgtaagcaaggtgctttgcagtgaaacctatgtgcaaagaaaaagcattttttatcatgcagaaacggagcgcctgctaactataggacatccatattacccagtgtctatcggggccaaaactgttcctaaggtctctgcaaatcagtatagggtatttaaaatacaactacctgatcccaatcaatttgcactacctgacaggactgttcacaacccaagtaaagagcggctggtgtgggcagtcataggtgtgcaggtgtccagagggcagcctcttggaggtactgtaactgggcaccccacttttaatgctttgcttgatgcagaaaatgtgaatagaaaagtcaccacccaaacaacagatgacaggaaacaaacaggcctagatgctaagcaacaacagattctgttgctaggctgtacccctgctgaaggggaatattggacaacagcccgtccatgtgttactgatcgtctagaaaatggcgcctgccctcctcttgaattaaaaaacaagcacatagaagatggggatatgatggaaattgggtttggtgcagccaacttcaaagaaattaatgcaagtaaatcagatctacctcttgacattcaaaatgagatctgcttgtacccagactacctcaaaatggctgaggacgctgctggtaatagcatgttcttttttgcaaggaaagaacaggtgtatgttagacacatctggaccagagggggctcggagaaagaagcccctaccacagatttttatttaaagaataataaaggggatgccacccttaaaatacccagtgtgcattttggtagtcccagtggctcactagtctcaactgataatcaaatttttaatcggccctactggctattccgtgcccagggcatgaacaatggaattgcatggaataatttattgtttttaacagtgggggacaatacacgtggtactaatcttaccataagtgtagcctcagatggaaccccactaacagagtatgatagctcaaaattcaatgtataccatagacatatggaagaatataagctagcctttatattagagctatgctctgtggaaatcacagctcaaactgtgtcacatctgcaaggacttatgccctctgtgcttgaaaattgggaaataggtgtgcagcctcctacctcatcgatattagaggacacctatcgctatatagagtctcctgcaactaaatgtgcaagcaatgtaattcctgcaaaagaagacccttatgcagggtttaagttttggaacatagatcttaaagaaaagctttctttggacttagatcaatttcccttgggaagaagatttttagcacagcaaggggcaggatgttcaactgtgagaaaacgaagaattagccaaaaaacttccagtaagcctgcaaaaaaaaaaaaaaaataa

SEQ ID NO: 5 - protein, 497 aa, Bovine papillomavirus type 2
MALWQQGQKLYLPPTPVSKVLCSETYVQRKSIFYHAETERLLTVGHPYYQVTVGDKTVPKVSANQFRVFKIQLPDPNQFALPDRTVHNPSKERLVWAVIGVQVSRGQPLGGTVTGHPTFNALLDAENVNRKVTAQTTDDRKQTGLDAKQQQILLLGCTPAEGEYWTTARPCVTDRLENGACPPLELKNKHIEDGDMMEIGFGAADFKTLNASKSDLPLDIQNEICLYPDYLKMAEDAAGNSMFFFARKEQVYVRHIWTRGGSEKEAPSKDFYLKNGRGEETLKIPSVHFGSPSGSLVSTDNQIFNRPYWLFRAQGMNNGIAWNNLLFLTVGDNTRGTNLSISVAADGNALSEYDTGKFNLYHRHMEEYKLAFILELCSVEITAQTLSHLQGLMPSVLQNWEIGVQPPASSILEDTYRYIESPATKCASNVIPPKEDPYAGLKFWSIDLKEKLSLDLDQFPLGRRFLAQQGAGCSTVRKRAVATRNSSKPAKRKKIKA

SEQ ID NO: 6 - DNA, 1494 bp, Bovine papillomavirus type 2
atggcgttgtggcaacaaggccaaaagctgtatctccctccaacccctgtaagcaaggtgctatgcagtgaaacctatgtgcaaagaaaaagcatattctatcatgcagaaacggaacgcctgttaactgtaggacatccatactaccaagtcactgtgggggacaaaactgttcccaaagtgtctgctaatcaatttagagtttttaaaatacagctccccgatcccaatcagtttgcattgcctgataggactgtgcacaatccaagcaaggagcgcctggtttgggctgtaataggggttcaagtatctcgtggccaaccactaggaggcacagttactgggcaccccacttttaatgctctgcttgatgcagaaaatgttaatagaaaagttactgcacaaacaacagatgacaggaagcaaacaggattagatgctaagcaacaacagattctgttgctgggctgtacccctgcagaaggggaatactggaccacagcccgtccatgtgttactgatagactagaaaatggtgcgtgtcctcctttagaattaaagaacaaacacatagaagatggagacatgatggaaatagggtttggtgctgctgactttaaaacactaaatgccagtaaatcagatctacctcttgacattcaaaatgaaatatgcctgtatccagactacctcaaaatggctgaagatgctgctggaaacagtatgttcttctttgcaagaaaagaacaagtgtatgtaaggcatatatggactcgggggggctctgaaaaagaagcacccagtaaagacttctacctcaaaaatggtagaggtgaagaaactctaaaaatacctagtgtgcactttggcagtcccagtggatccttggtgtccactgataatcaaatatttaacaggccttattggctattcagggctcagggcatgaacaatgggattgcatggaataatttattatttttaactgtaggggataacacacggggaactaaccttagtattagtgtagctgcagatggaaacgcattgtcagagtatgatactggcaaatttaacctataccataggcatatggaagaatataagctagcatttatattggagctgtgctctgttgagattactgcacaaacactgtcacatctgcaaggactgatgccctctgtgctacaaaactgggaaatcggggtgcaacctcctgcttcttctattttagaagatacttataggtacatagagtctcctgcaactaaatgtgcaagtaatgttataccacccaaagaagacccttatgcagggcttaagttttggagcatagacttaaaagaaaagctgtctttggacttagaccaatttcccttgggaagaagattcttagctcagcaaggggcaggatgttcaactgtgagaaagagagctgttgcaaccagaaattccagtaagcctgcaaaaagaaaaaaaatcaaagcttaa

(19) United States
(12) Patent Application Publication
(10) Pub. No.: US 2009/0011996 A1
(43) Pub. Date: Jan. 8, 2009

(54) DIAGNOSING AND PROTECTING HORSES AGAINST PAPILLOMAVIRUS

(75) Inventors: A. Bennett Jenson, Louisville, KY (US); Shin-je Ghim, Louisville, KY (US); Victoria B. Kyle, Louisville, KY (US); Joongho Joh, Louisville, KY (US)

Correspondence Address: STITES & HARBISON, PLLC, 400 W MARKET ST, SUITE 1800, LOUISVILLE, KY, 40202-3352, US

(73) Assignee: UNIVERSITY OF LOUISVILLE RESEARCH FOUNDATION, INC., Louisville, KY (US)

(21) Appl. No.: 12/167,581

(22) Filed: Jul. 3, 2008

Related U.S. Application Data
(60) Provisional application No. 60/948,315, filed on Jul. 6, 2007.

(51) Int. Cl. A61K 38/16; C12Q 1/70; A61P 31/12

(52) U.S. Cl. 514/12; 435/5

(57) ABSTRACT

Compositions for conferring protection against Equus caballus papillomavirus (EcPV) and Bovine papillomavirus (BPV) infection in a subject are provided and include a virus-like particle assembled from an EcPV L1 protein and virus like particle assembled from a BPV L1 protein. Methods for protecting a subject against EcPV and BPV infection are further provided and include administering to the subject a composition comprised of a virus-like particle assembled from an EcPV L1 protein and virus like particle assembled from a BPV L1 protein. Methods of diagnosing EcPV and/or BPV infection in a subject are also provided and include providing a virus-like particle assembled from a papillomavirus L1 protein selected from an EcPV L1 protein and a BPV L1 protein; contacting the virus-like particle with serum from the subject; and identifying the subject as having an infection if an antibody capable of binding the virus-like particle is detected in the serum.

### RELATED APPLICATIONS

This application claims priority from U.S. Provisional Application Ser. No. 60/948,315 filed Jul. 6, 2007, which is incorporated herein by this reference.

### TECHNICAL FIELD

The presently-disclosed subject matter relates to diagnosis and prevention of papillomavirus infections in animals, and, more particularly, to diagnosis and prevention of papillomavirus infections in horses.

### INTRODUCTION AND GENERAL CONSIDERATIONS

Papillomaviruses have been a plague of domesticated horses for centuries. The most common cutaneous tumor of horses is papillomatosis in yearlings, covering the face and adjacent areas with small, elevated, circumscribed horny masses. Equus caballus papillomavirus type 1 (EcPV-1) has been identified as the causative agent of this disease. These benign and persistent tumors are an irritation to horses, and are also undesirable because they affect the value of the horses and infected horses are barred from public shows and sales as yearlings. Papillomas on the muzzle can persist for 1-9 months and then regress spontaneously. Papillomas covering other areas, such as the ears, do not regress.

In general, papillomaviruses are species-specific and interspecies transmission is a rare event; however, horses can become infected with bovine papillomavirus (BPV). Bovine papillomavirus types 1 and 2 (BPV-1 and -2) are responsible for most, if not all, equine sarcoid. Sarcoid is the second most common tumor in horses and is a PV-induced lesion causing a ½ grade sarcoma. This is a dermatological locally aggressive neoplasm with no consistently effective therapy.

Unlike equine papillomatosis, equine sarcoid is not a productive infection and, therefore, infectious BPV cannot be transmitted from one horse to another. Rubbing of horses and infected cattle on barb wire fences separating the two species is a frequent mode of transmission from cattle to horses. Also, even if cattle are injected with BPV-1, and BPV-1 does not come into contact with cutaneous epithelium, it can cause meningiomas, bladder tumors and other tumors of fibroblastic origin.

The fibromatosis of equine sarcoid can extend into the capsule of joints and into essential muscles of the affected animals resulting in the discomfort of the horse and loss of service and value to the owner. There is no effective treatment, and the sarcoid does not always regress spontaneously. Because of this, the infected horses usually have to be destroyed.

Accordingly, there remains a need in the art for compositions and methods for protecting horses against Equus caballus papillomavirus and Bovine papillomavirus.

### SUMMARY

The presently-disclosed subject matter meets some or all of the above-identified needs, as will become evident to those of ordinary skill in the art after a study of information provided in this document. This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes compositions comprising virus-like particles (VLPs) assembled from an L1 protein of at least one type of Equus caballus papillomavirus (EcPV) and virus-like particles (VLPs) assembled from an L1 protein of at least one type of Bovine papillomavirus (BPV).

In some embodiments, the composition can include a VLP of an L1 protein of EcPV, where the EcPV is EcPV type 1 (EcPV-1). In some embodiments, the composition can include a VLP of an L1 protein of BPV, where the BPV is BPV type 1 (BPV-1). In some embodiments, the composition can include a VLP of an L1 protein of BPV, where the BPV is BPV type 2 (BPV-2). In some embodiments, the composition can include a VLP of an L1 protein of EcPV-Protein 1 and a VLP of an L1 protein of BPV-1. In some embodiments, the immunogenic composition can include a VLP of an L1 protein of EcPV-1 and a VLP of an L1 protein of BPV-2. In some embodiments, the immunogenic composition can include a VLP of an L1 protein of EcPV-1, a VLP of an L1 protein of BPV-1, and a VLP of an L1 protein of BPV-2.

In some embodiments, the VLP of the L1 protein of EcPV is assembled from a functional polypeptide that is encoded by a nucleic acid molecule comprising (a) the sequence of SEQ ID NO: 2, (b) a degenerate variant of SEQ ID NO: 2, or (c) a fragment of SEQ ID NO: 2.

In some embodiments, the VLP of the L1 protein of EcPV is assembled from a functional polypeptide selected from a polypeptide comprising (a) the sequence of SEQ ID NO: 1, (b) a fragment of SEQ ID NO: 1, or (c) a polypeptide comprising the sequence of SEQ ID NO: 1 with about 1, 2, 3, 4, or 5 conservative amino acid substitutions. In some embodiments, the VLP of the L1 protein of EcPV is assembled from a functional polypeptide comprising a fragment of SEQ ID NO: 1 where the fragment comprises about amino acid 1 to about amino acid 480 of SEQ ID NO: 1. In some embodiments, the VLP of the L1 protein of EcPV is assembled from a functional polypeptide comprising a fragment of SEQ ID NO: 1 where the fragment comprises about amino acid 1 to about amino acid 479 of SEQ ID NO: 1.

In some embodiments, the VLP of the L1 protein of BPV is assembled from a functional polypeptide that is encoded by a nucleic acid molecule comprising (a) the sequence of SEQ ID NO: 4; (b) a degenerate variant of SEQ ID NO: 4; or (c) a fragment of SEQ ID NO: 4.

In some embodiments, the VLP of the L1 protein of BPV is assembled from a functional polypeptide selected from a polypeptide comprising (a) the sequence of SEQ ID NO: 3; (b) a fragment of SEQ ID NO: 3; or (c) a polypeptide comprising the sequence of SEQ ID NO: 3 with about 1, 2, 3, 4, or 5 conservative amino acid substitutions. In some embodiments, the VLP of the L1 protein of BPV is assembled from a functional polypeptide comprising a fragment of SEQ ID NO: 3 where the fragment comprises about amino acid 1 to about amino acid 470 of SEQ ID NO: 3. In some embodiments, the VLP of the L1 protein of BPV is assembled from a functional polypeptide comprising a fragment of SEQ ID NO: 3 where the fragment comprises about amino acid 1 to about amino acid 469 of SEQ ID NO: 3.

In some embodiments, the VLP of the L1 protein of BPV is assembled from a functional polypeptide that is encoded by a nucleic acid molecule comprising (a) the sequence of SEQ ID NO: 6; (b) a degenerate variant of SEQ ID NO: 6; or (c) a fragment of SEQ ID NO: 6.

In some embodiments, the VLP of the L1 protein of BPV is assembled from a functional polypeptide comprising (a) the sequence of SEQ ID NO: 5; (b) a fragment of SEQ ID NO: 5; or (c) a polypeptide comprising the sequence of SEQ ID NO: 5 with up to about 1, 2, 3, 4, or 5 conservative amino acid substitutions. In some embodiments, the VLP of the L1 protein of BPV is assembled from a functional polypeptide comprising a fragment of SEQ ID NO: 5 where the fragment comprises about amino acid 1 to about amino acid 472 of SEQ ID NO: 5. In some embodiments, the VLP of the L1 protein of BPV is assembled from a functional polypeptide comprising a fragment of SEQ ID NO: 5 where the fragment comprises about amino acid 1 to about amino acid 471 of SEQ ID NO: 5.

In some embodiments, the composition further includes an adjuvant.

The presently-disclosed subject matter includes methods of protecting a subject against EcPV and BPV infection by administering a composition of the presently-disclosed subject matter.

The presently-disclosed subject matter includes methods of diagnosing EcPV and/or BPV infection in a subject, including: providing a virus-like particle assembled from EcPV L1 protein and/or BPV L1 protein; contacting the virus-like particle with serum obtained from the subject; and identifying the subject as having an infection if an antibody capable of binding the virus-like particle is detected in the serum.

In some embodiments, the binding is detected using an antibody capable of binding the EcPV antibody, and an antibody capable of binding the BPV antibody.

### BRIEF DESCRIPTION OF THE DRAWINGS

FIG. 1 is a linear representation of the open reading frames (ORFs) of the EcPV-1 genome depicting the early and late proteins, and the upstream regulatory region (URR).

FIG. 2 is a diagram depicting the production of VLPs using recombinant baculovirus vectors to express the L1 proteins in eukaryotic cells.

FIG. 3 is a transmission electron microscopy picture of negatively stained, purified BPV-1 VLPs.

FIG. 4 is a transmission electron microscopy picture of negatively stained, purified EcPV-1 VLPs.

FIG. 5 is a graph of the antibody titer against VLPs of EcPV as a function of time in subjects receiving an initial administration at 0 weeks and receiving a booster administration at 2 weeks of either an EcPV composition (), an EcPV and BPV-1 divalent composition (_), or a BPV-1 composition (▪).

FIG. 6 is a graph of the antibody titer against BPV-1 as a function of time in subjects receiving an initial administration at 0 weeks and receiving a booster administration at 2 weeks of either an EcPV composition (), an EcPV and BPV-1 divalent composition (_), or a BPV-1 composition (▪).

### BRIEF DESCRIPTION OF THE SEQUENCE LISTING

SEQ ID NO: 1 is an amino acid sequence of an L1 polypeptide of Equus caballus papillomavirus type 1 (EcPV-1);

SEQ ID NO: 2 is a nucleic acid sequence that encodes an L1 polypeptide of Equus caballus papillomavirus type 1 (EcPV-1);

SEQ ID NO: 3 is an amino acid sequence of an L1 polypeptide of Bovine papillomavirus type 1 (BPV-1);

SEQ ID NO: 4 is a nucleic acid sequence that encodes an L1 polypeptide of Bovine papillomavirus type 1 (BPV-1);

SEQ ID NO: 5 is an amino acid sequence of an L1 polypeptide of Bovine papillomavirus type 2 (BPV-2); and

SEQ ID NO: 6 is a nucleic acid sequence that encodes an L1 polypeptide of Bovine papillomavirus type 2 (BPV-2).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described. While the following terms are believed to be well understood by one of ordinary skill in the art, the following definitions are set forth to facilitate explanation of the presently-disclosed subject matter.

Following long-standing patent law convention, the terms “a”, “an”, and “the” refer to “one or more” when used in this application, including the claims. Thus, for example, reference to “a VLP” includes a plurality of such VLPs, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration, or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods.

The term “associated with”, and “operatively linked” refer to two nucleic acid sequences that are related physically or functionally. For example, a promoter or regulatory DNA sequence is said to be “associated with” a DNA sequence that encodes an RNA or a polypeptide if the two sequences are operatively linked, or situated such that the regulator DNA sequence will affect the expression level of the coding or structural DNA sequence.

The terms “coding sequence” and “open reading frame” (ORF) are used interchangeably and refer to a nucleic acid sequence that is transcribed into RNA such as mRNA, rRNA, tRNA, snRNA, sense RNA, or antisense RNA. In some embodiments, the RNA is then translated in vivo or in vitro to produce a polypeptide.

The term “conservatively-substituted variant” refers to a peptide comprising an amino acid residue sequence that differs from a reference peptide by one or more conservative amino acid substitution, and maintains some or all of the activity of the reference peptide as described herein. A “conservative amino acid substitution” is a substitution of an amino acid residue with a functionally similar residue. Examples of conservative substitutions include the substitution of one non-polar (hydrophobic) residue such as alanine, isoleucine, valine, leucine, or methionine for another; the substitution of one polar (hydrophilic) residue for another such as between arginine and lysine, between glutamine and asparagine, between glycine and serine; the substitution of one basic residue such as lysine, arginine, or histidine for another; or the substitution of one acidic residue, such as aspartic acid or glutamic acid for another. The phrase “conservatively-substituted variant” also includes peptides wherein a residue is replaced with a chemically-derivatized residue, provided that the resulting peptide maintains some or all of the activity of the reference peptide as described herein.

The term “degenerate variant” refers to a nucleic acid having a residue sequence that differs from a reference nucleic acid by one or more degenerate codon substitutions. Degenerate codon substitutions can be achieved by generating sequences in which the third position of one or more selected (or all) codons is substituted with mixed-base and/or deoxyinosine residues (See e.g., Batzer et al. (1991) Nucleic Acid Res 19:5081; Ohtsuka et al. (1985) J Biol Chem 260:2605-2608; Rossolini et al. (1994) Mol Cell Probes 8:91-98).

As used herein, the term “effective amount” refers to a dosage sufficient to provide protection against papillomavirus infection. As such, in some embodiments, the term “effective amount” can refer to a dosage sufficient to provide an antibody response that will confer protection against papillomavirus infection. The exact amount that is required will vary from subject to subject, depending on the species, age, and general condition of the subject, the particular adjuvant being used, mode of administration, and the like. As such, the effective amount will vary based on the particular circumstances, and an appropriate effective amount can be determined in a particular case by one of ordinary skill in the art using only routine experimentation.

The term “expression vector” refers to a bacteriophage, a plasmid, or another like agent, containing an expression cassette, comprising a nucleic acid molecule capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The coding region usually encodes a polypeptide of interest but can also encode a functional RNA of interest, for example antisense RNA or a non-translated RNA, in the sense or antisense direction. The expression cassette comprising the nucleotide sequence of interest can be chimeric, meaning that at least one of its components is heterologous with respect to at least one of its other components. The expression cassette can also be one that is naturally-occurring but has been obtained in a recombinant form useful for heterologous expression.

Typically, however, the expression cassette is heterologous with respect to the host; i.e., the particular DNA sequence of the expression cassette does not occur naturally in the host cell and was introduced into the host cell or an ancestor of the host cell by a transformation event. The expression of the nucleotide sequence in the expression cassette can be under the control of a constitutive promoter or of an inducible promoter that initiates transcription only when the host cell is exposed to some particular external stimulus.

The term “fragment” refers to a nucleic acid or amino acid sequence that comprises a subset of another nucleic acid or amino acid sequence. A fragment of a nucleic acid sequence can be any number of nucleotides that is less than that found in another nucleic acid sequence, and thus includes, but is not limited to, the sequences of an exon or intron, a promoter, an enhancer, an origin of replication, a 5′ or 3′ untranslated region, a coding region, and a polypeptide binding domain. It is understood that a fragment can also comprise less than the entirety of a nucleic acid sequence, for example, a portion of an exon or intron, promoter, enhancer, etc. Similarly, a fragment of an amino acid sequence can refer to a polypeptide in which amino acid residues are deleted as compared to a reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long.

A fragment can retain one or more of the biological activities of the reference polypeptide. In some embodiments, a fragment can comprise a domain or feature, and optionally additional amino acids on one or both sides of the domain or feature, which additional amino acids can number from 5, 10, 15, 20, 30, 40, 50, or up to 100 or more residues. Further, fragments can include a sub-fragment of a specific region, which sub-fragment retains a function of the region from which it is derived.

The term “functional”, when used in reference to a polypeptide, e.g. a full-length protein or fragment thereof, refers to a polypeptide against which antibodies effective for protecting against infection can be generated. In some embodiments, the functional polypeptide can be the full-length amino acid sequence of a reference polypeptide. In some embodiments, the functional polypeptide can comprise a polypeptide fragment of a reference polypeptide. In some embodiments, the functional polypeptide can comprise a polypeptide fragment of a reference polypeptide that retains the protein confirmation and epitopes of the full-length reference polypeptide.

The term “gene” is used broadly to refer to any segment of DNA associated with a biological function. Thus, genes include, but are not limited to, coding sequences and/or the regulatory sequences required for their expression. Genes can also include non-expressed DNA segments that, for example, form recognition sequences for a polypeptide. Genes can be obtained from a variety of sources, including cloning from a source of interest or synthesizing from known or predicted sequence information, and can include sequences designed to have desired parameters.

The terms “heterologous”, “recombinant”, and “exogenous”, when used herein to refer to a nucleic acid sequence (e.g. a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found. Similarly, when used in the context of a polypeptide or amino acid sequence, an exogenous polypeptide or amino acid sequence is a polypeptide or amino acid sequence that originates from a source foreign to the particular host cell or, if from the same source, is modified from its original form. Thus, exogenous DNA segments can be expressed to yield exogenous polypeptides. A “homologous” nucleic acid (or amino acid) sequence is a nucleic acid (or amino acid) sequence naturally-associated with a host cell into which it is introduced.

The term “isolated”, when used in the context of an isolated nucleic acid molecule or an isolated polypeptide, is a DNA molecule or polypeptide that, by the hand of man, exists apart from its native environment and is therefore not a product of nature. An isolated DNA molecule or polypeptide can exist in a purified form or can exist in a non-native environment such as, for example, in a transgenic host cell.

The term “nucleic acid” refers to deoxyribonucleotides or ribonucleotides and polymers thereof in either single-or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues. The terms “nucleic acid” or “nucleic acid sequence” can also be used interchangeably with gene, open reading frame (ORF), cDNA, and mRNA encoded by a gene.

The terms “polypeptide”, “protein”, and “peptide”, which are used interchangeably herein, refer to a polymer of the 20 protein amino acids, or amino acid analogs, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

As used herein, the terms “protecting” or “protection” refer to preventing or inhibiting the spread of a pathogenic infection, such as a papillomavirus infection. As such, “protecting” or “protection” can include at least the partial prevention of the symptoms associated with a pathogenic infection and/or its complications. In some embodiments, preventing or inhibiting the spread of infection occurs because a polypeptide or virus-like particle assembled from the polypeptide elicits an immunological response that is specific for an Equus caballus papillomavirus (EcPV) and/or a bovine papillomavirus (BPV). Such a response can be cellular or humoral. Thus, the stimulation of antibodies, T-cells, macrophages, B-cells, dendritic cells, etc., by a polypeptide or virus like particle assembled from the polypeptide, e.g., polypeptides of SEQ ID NOs: 1, 3, and 5, can protect against EcPV or BPV infection. These responses can be measured routinely, as will be understood by one of ordinary skill in the art.

The term “transformation” refers to a process for introducing heterologous DNA into a cell. Transformed cells are understood to encompass not only the end product of a transformation process, but also transgenic progeny thereof.

The presently-disclosed subject matter includes compositions and methods related to diagnosing and protecting against Equus caballus papillomavirus (EcPV) and Bovine papillomavirus (BPV).

The PV genes and associated proteins can be classified with respect to their chronological appearance during the viral life cycle. In this regard, they can be classified as early (E) and late (L), early referring to a time before replication of the virus has begun, and late referring to a time after replication of the virus has begun. PVs contain a covalently-closed circular DNA double strand with open reading frames (ORFs) located on the coding strand. Products of late (L) genes represent the structural capsid proteins, whereas products of the two early (E) genes fulfill regulatory tasks during cell transformation, replication, and transcription. The presently-disclosed subject matter includes compositions comprising L1 papillomavirus proteins.

The presently-disclosed subject matter includes compositions and vaccines comprising virus-like particles (VLPs) assembled from an L1 protein of at least one type of Equus caballus papillomavirus (EcPV), and a VLP assembled from an L1 protein of at least one type of Bovine papillomavirus (BPV). In certain embodiments, L1-VLPs of one type of EcPV and one type of BPV are provided, e.g., EcPV Type 1 (EcPV-1) and BPV Type 1 (BPV-1). In other embodiments, L1 -VLPs of more than one type of EcPV and/or more than one type of BPV can be provided, e.g., EcPV-1, BPV-1, and BPV Type 2 (BPV-2).

As used herein, an L1 protein of an EcPV or an L1 protein of a BPV refers to a full-length L1 protein, or a fragment thereof that retains the conformation and epitopes of the full-length L1 protein, i.e., functional polypeptides. For example, in certain embodiments, a full-length L1 protein of EcPV-1, BPV-1, and/or BPV-2 can be used. For another example, in certain embodiments, a fragment of the L1 protein of EcPV-1, BPV-1, and/or BPV-2 can be used.

Exemplary functional polypeptides of the L1 protein of EcPV-1 include, for example, fragments of the L1 protein wherein up to about 26 amino acids are removed from the C-terminus, up to about 25 amino acids are removed from the C-terminus, up to about 20 amino acids are removed from the C-terminus, up to about 15 amino acids are removed from the C-terminus, up to about 10 amino acids are removed from the C-terminus, up to about 5 amino acids are removed from the C-terminus, or about 1 amino acid is removed from the C-terminus, relative to the full-length L1 protein. In some embodiments, the L1 protein of EcPV-1 can be a functional polypeptide comprising a fragment of SEQ ID NO: 1 where the fragment comprises about amino acid 1 to about amino acid 480 of SEQ ID NO: 1. In some embodiments, the L1 protein of EcPV-1 can be a functional polypeptide comprising a fragment of SEQ ID NO: 1 where the fragment comprises about amino acid 1 to about amino acid 479 of SEQ ID NO: 1.

Exemplary functional polypeptides of the L1 protein of BPV-1 include, for example, fragments of the L1 protein wherein up to about 26 amino acids are removed from the C-terminus, up to about 25 amino acids are removed from the C-terminus, up to about 20 amino acids are removed from the C-terminus, up to about 15 amino acids are removed from the C-terminus, up to about 10 amino acids are removed from the C-terminus, up to about 5 amino acids are removed from the C-terminus, or about 1 amino acid is removed from the C-terminus, relative to the full-length L1 protein. In some embodiments, the L1 protein of BPV can be a functional polypeptide comprising a fragment of SEQ ID NO: 3 where the fragment comprises about amino acid 1 to about amino acid 470 of SEQ ID NO: 3 or about amino acid 1 to about amino acid 469 of SEQ ID NO: 3.

Exemplary functional polypeptides of the L1 protein of BPV-2 include, for example, fragments of the L1 protein wherein up to about 26 amino acids are removed from the C-terminus, up to about 25 amino acids are removed from the C-terminus, up to about 20 amino acids are removed from the C-terminus, up to about 15 amino acids are removed from the C-terminus, up to about 10 amino acids are removed from the C-terminus, up to about 5 amino acids are removed from the C-terminus, or about 1 amino acid is removed from the C-terminus, relative to the full-length L1 protein. In some embodiments, the L1 protein of BPV can be a functional polypeptide comprising a fragment of SEQ ID NO: 5 where the fragment comprises about amino acid 1 to about amino acid 472 of SEQ ID NO: 5. In some embodiments, the L1 protein of BPV can be a functional polypeptide comprising a fragment of SEQ ID NO: 5 where the fragment comprises about amino acid 1 to about amino acid 471 of SEQ ID NO: 5.

Polypeptide fragments described herein can be used as immunogens for raising antibodies that can bind to the full-length L1 protein. Such antibodies can be used in detection and isolation methods, as would be understood by one of ordinary skill in the art.

Also, as used herein, an L1 protein of an EcPV refers to an isolated, functional polypeptide, comprising a polypeptide including the sequence of SEQ ID NO: 1 with about 1, 2, 3, 4, or 5 conservative amino acid substitutions.

Also, as used herein, an L1 protein of a BPV refers to an isolated, functional polypeptide, comprising a polypeptide including the sequence of SEQ ID NO: 3 or SEQ ID NO: 5 with about 1, 2, 3, 4, or 5 conservative amino acid substitutions.

As used herein, an L1 protein of an EcPV also refers to an isolated, functional polypeptide being encoded by a nucleic acid molecule including the sequence of SEQ ID NO: 2, or a fragment or a degenerate variant thereof.

Also, as used herein, an L1 protein of a BPV refers to an isolated, functional polypeptide being encoded by a nucleic acid molecule including the sequence of SEQ ID NO: 4, or a fragment or a degenerate variant thereof.

Also, as used herein, an L1 protein of a BPV refers to an isolated, functional polypeptide being encoded by a nucleic acid molecule including the sequence of SEQ ID NO: 6, or a fragment or a degenerate variant thereof.

An exemplary process for preparing EcPV L1 VLPs and/or BPV L1 VLPs makes use of an expression system including an expression vector and an appropriate host cell. The expression vector includes a PV L1 nucleotide sequence capable of encoding a PV L1 protein of interest. For example, when the protein of interest is the EcPV-1 L1 protein, the expression vector can include the nucleotide sequence of SEQ ID NO: 2, or a fragment or a degenerate variant thereof, which is capable of encoding an EcPV-1 L1 protein (full length or functional fragment). For another example, when the protein of interest is the BPV-1 L1 protein, the expression vector can include the nucleotide sequence of SEQ ID NO: 4, or a fragment or a degenerate variant thereof, which is capable of encoding a BPV-1 L1 protein (full length or functional fragment). For another example, when the protein of interest is the BPV-2 L1 protein, the expression vector can include the nucleotide sequence of SEQ ID NO: 6, or a fragment or a degenerate variant thereof, which is capable of encoding a BPV-1 L1 protein (full length or functional fragment).

The host cell is infected with the vector. Recombinant L1 proteins are generated and self-assemble into VLPs in the host cell. The resulting VLPs are isolated and purified. Additional information related to methods of producing PV proteins of interest and VLPs, including PV L1 proteins and VLPs, can be found in Examples presented in this document, and in the following references, each of which is incorporated herein by reference: U.S. Pat. Nos. 5,057,411; 5,874,089; 6,485,728; 6,887,478; 7,001,995; 6,908,615; 6,165,471; and 6,153,201; and United States Patent Application Publication Nos. 2002/0197264; 2004/0086527; 2005/0026257; 2006/0029612; and 2005/0282263.

In certain embodiments, the compositions and vaccines can include VLPs assembled from at least one EcPV L1 protein and at least one BPV L1 protein provided in a pharmaceutically-acceptable formulation. Suitable formulations include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. The compositions can take such forms as suspensions, solutions, or emulsions in oily or aqueous vehicles, and can contain formulatory agents such as suspending, stabilizing, and/or dispersing agents. The formulations can be presented in unit-dose or multi-dose containers, for example sealed ampoules and vials, and can be stored in a frozen or freeze-dried (lyophilized) condition requiring only the addition of sterile liquid carrier immediately prior to use.

In certain embodiments, the compositions can include VLPs assembled from at least one EcPV L1 protein and at least one BPV L1 protein, and an adjuvant. Suitable adjuvants for use in the practice of the present subject matter include, but are not limited to (1) polymers of acrylic or methacrylic acid, maleic anhydride and alkenyl derivative polymers, (2) immunostimulating sequences (ISS), such as oligodeoxyribonucleotide sequences having one or more non-methylated CpG units (Klinman et al., Proc. Natl. Acad. Sci., USA, 1996, 93, 2879-2883; WO98/16247), (3) an oil in water emulsion, such as the SPT emulsion described on p 147 of “Vaccine Design, The Subunit and Adjuvant Approach” published by M. Powell, M. Newman, Plenum Press 1995, and the emulsion MF59 described on p 183 of the same work, (4) cation lipids containing a quaternary ammonium salt, (5) cytokines, (6) aluminum hydroxide or aluminum phosphate, (7) other adjuvants discussed in any document cited and incorporated by reference into this document, or (8) any combinations or mixtures thereof.

The presently-disclosed subject matter includes a method of protecting a subject against EcPV and BPV infection by administering VLPs assembled from at least one EcPV L1 protein and at least one BPV L1 protein. In certain embodiments, the method can include protecting a subject against EcPV and BPV infection by administering a composition or vaccine, as described above. As used herein, the term “subject” includes any animal capable of being infected by EcPV and BPV. In some embodiments, the composition or vaccine can be administered to any herd of subjects.

Equine papillomatosis is a highly contagious PV infection of yearlings that results in papillomas that produce neutralizing antibodies when they regress, and as many as 75% of mature horses have been previously infected with naturally occurring PV virions. Current United States Department of Agriculture (USDA) rules and regulations, however, generally only permit vaccines or compositions to be used to protect the particular herds from which the organisms used for producing the vaccine were isolated. These rules and regulations are to ensure that the vaccines will be protective in the case of RNA viruses or higher microorganisms that are known to mutate due to antigenic drift. DNA viruses, however, such as EcPV and BPV, generally do not exhibit substantial mutation. As such, a single composition or vaccine formulation against EcPV and/or BPV infection could be used worldwide to protect against EcPV and/or BPV infection.

It is appreciated that a naturally-occurring infection by PV virions or administration of a PV vaccine comprised of VLPs produces a neutralizing antibody response against conformationally-dependent, immunodominant epitopes on the surface of the L1 capsid protein of a PV. If an animal has been previously infected with a PV, the animal retains low titer of the neutralizing antibodies against PV, with protection against later infection being provided by the low titer of neutralizing antibodies and circulating immune cells that respond to a later PV infection with a secondary immune response.

Administration of a vaccine or composition of the presently-disclosed subject matter produces a secondary immune response in animals that have been previously infected with an EcPV and/or a BPV, thus indicating that the VLPs of the presently-disclosed subject matter present the same antigenic determinants as naturally-occurring PV virions and induce an immune response to immunodominant, conformationally-dependent, neutralizing epitopes. In some embodiments, the VLPs of the presently-disclosed subject matter present substantially identical antigenic determinants as those found on naturally-occurring PV virions. As such, the VLPs of the presently-disclosed subject matter will induce a secondary immune response in mature horses if there has been a previous infection with naturally-occurring virions, and the VLPs will induce a primary immune response in yearlings when used for vaccination of animals that have not been challenged with the naturally-occurring virions.

For example, administration of a composition or vaccine of the presently-disclosed subject matter to older horses that were infected with an EcPV as yearlings, followed by an observation of a secondary immune response, indicates that the VLPs present the same immunodominant, conformationally-dependent epitopes found on the surface of the L1 capsid protein of naturally-occurring EcPV virions. As such, the presently-disclosed subject matter allows for a determination of whether mature horses from different herds throughout the United States have been infected with a naturally-occurring EcPV containing a capsid that has substantially identical, neutralizing epitopes as the presently-disclosed VLPs. A finding that EcPV virions have infected mature horses of herds throughout the United States when they are yearlings, and a finding that the infectious EcPV virion, that naturally infected the horses, has a capsid that mimics the VLPs, thus indicates that only one vaccine VLP formulation is necessary for vaccinating yearlings of all herds.

The compositions disclosed herein can be formulated for administration, as will be understood by those of ordinary skill in the art. Techniques and formulations generally can be found in Remington's Pharmaceutical Sciences, Meade Publishing Co., Easton, Pa. Exemplary methods of administration will be understood by those of ordinary skill in the art, and include parenteral administration, e.g., intravascular injection, such as intravenously (IV) or intraarterially, or intramuscular injection. Administration protocols can be optimized using procedures generally known in the art. A single dose can be administered to a subject, or alternatively, two or more inoculations can take place with intervals of several days, weeks, or months.

The presently-disclosed subject matter includes a method for diagnosing EcPV and/or BPV infection in a subject. In certain embodiments, the method includes providing an EcPV L1 -VLP and/or a BPV L1-VLP; contacting the EcPV L1-VLP and/or a BPV L1-VLP with serum obtained from the subject; and identifying the subject as having EcPV and/or BPV infection if an antibody capable of binding the EcPV L1-VLP and/or a BPV L1-VLP is detected in the serum.

Binding between L1-VLPs can be detected using a tagged-antibody capable of binding to the VLP antibody. Alternatively, binding between L1-VLPs can be detected using a series of antibodies, wherein at least one antibody in the series binds to the VLP antibodies, and at least one antibody in the series is tagged for detection. For example, an appropriate series of antibodies can include a primary antibody capable of binding the VLP antibody, and a secondary antibody capable of binding the primary antibody, which secondary antibody is tagged to allow for detection, e.g., fluorescent, radioactive, etc.

In certain embodiments, the method includes providing an EcPV L1-VLP and/or a BPV L1 -VLP immobilized on a substrate; for example, an EL1 SA plate (Dynatech Laboratories, Inc., Chantilly, Va.) can be coated with L1 -VLPs of at least one type of EcPV and/or at least one type of BPV. In certain embodiments, L1-VLP of different types of EcPV and/or BPV can be immobilized on a substrate in an organized manner, such that diagnosis of individual and/or multiple types of ECPV and/or BPV can be readily made, as will be understood by those of ordinary skill in the art upon reviewing this document. In certain embodiments, various controls can be provided, which can be desirable for use in comparing binding detection results for test serum, as will be understood by those of ordinary skill in the art upon reviewing this document. Additional information related to methods for diagnosing EcPV and BPV can be found in Examples presented in this document, and information related to detecting antibody/PV L1 -Protein binding can be found in U.S. patent application Ser. Nos. 6,887,478; 6,485,728; and 5,874,089, which are incorporated herein by reference.

The presently-disclosed subject matter is further illustrated by the following specific but non-limiting examples. Some of the following examples are prophetic. Some of the following examples may include compilations of data that are representative of data gathered at various times during the course of development and experimentation related to the present invention.

### EXAMPLES

### Example 1

### DNA Extraction

Samples were taken from an equine cutaneous papilloma, and a bovine wart. Tissue biopsies were finely minced with a scalpel and digested overnight at 55° C. in digestion buffer (10 mM Tris, 0.5% SDS, pH 7.4) containing 500 μg proteinase K. Deproteinization is performed by phenol-, phenol-chloroform-isoamylalcohol-, and chloroform-extractions followed by ethanol precipitation to recover DNA. Air-dried DNA-pellets are then resuspended in 20-50 μl TE-buffer (10 mM Tris-HCl, 1 mM EDTA, pH 8.0).

### Example 2

### Isothermal Multiply Primed RCA

To amplify PV-DNA isolated from tissues, a rolling-circle-amplification (RCA) was carried out with the TempliPhi™ 100 Amplification Kit (Amersham Biosciences, Roosendaal, The Netherlands) following the manufacturer's instructions using 1-2 μl of extracted DNA and adding 450 μM extra dNTPs as previously described in Rector, et al. 2004 Journal of Virology 78:12698-12702, which is incorporated herein by reference. Restriction enzyme-digested amplified products were then examined in agarose gels, and right-sized products were cloned and sequenced.

### Example 3

### DNA Cloning and Sequencing

The entire isolated, RC-amplified PV genomes of different types were cloned into appropriate vectors via appropriate restriction sites. The genomes were sequenced using transposon integrations with the EZ::TN <KAN-2> Insertion Kit (Epicentre, Madison, Wis., USA) according to the manufacturer's protocol. Multiple colonies representing multiple integration sites were sequenced forward and backward. The sequences are put together using appropriate software, e.g., DNASTAR Lasergene SeqMan software (version 5.52). See Ghim, et al., Biochem Biophys Res Comm. Nov. 19, 2004; 324 (3): 1108-1115, which is incorporated herein by this reference.

### Example 4

### DNA and Protein Sequence Analysis

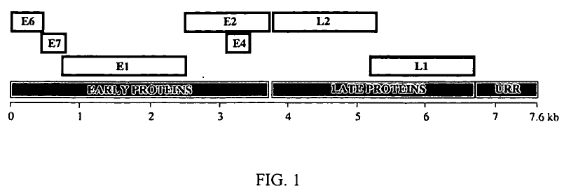

ORFs (FIG. 1) were identified using MacVector (version 7.2), and nucleotide and protein sequence similarities were searched via the NCBI BLAST server. Sequence alignments and the Phylogeny were calculated with DNASTAR Lasergene SeqMan, ClustalW, GENEDOC and MEGA3.

### Example 5

### VLP Production

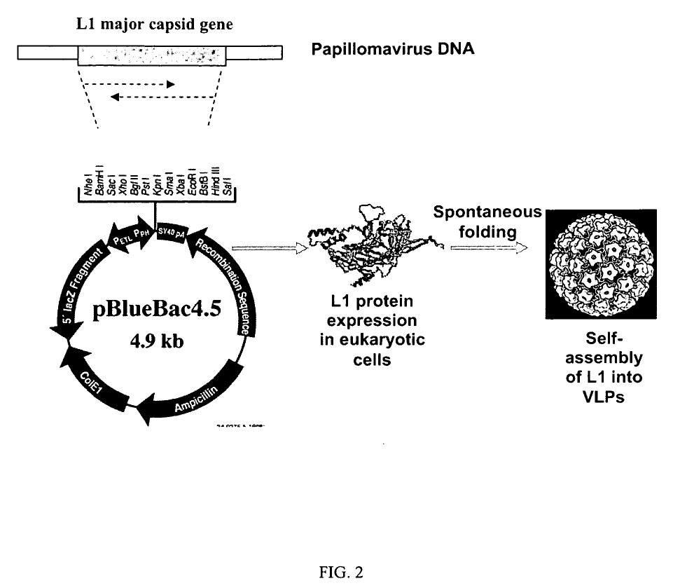

Using cloned complete PV templates, the corresponding L1 genes encoding the L1 proteins were amplified using polymerase chain reaction (PCR). Appropriate fragments were isolated and separately inserted into the multiple cloning site of the baculoviral transfer vector (FIG. 2). Recombinant baculovirus vectors were prepared using the Bac-N-Blue Kit (Invitrogen, Carlsbad, Calif., USA), according to the manufacturer's instructions, to transfect appropriate insect cells as described in Ghim, S. et al., Biochem Biophys Res Comm. Nov. 19, 2004; 324 (3): 1108-1115, which is incorporated herein by reference.

The insect cells were cultured in supplemented Grace's medium (Gibco/BRL, Gaithersburg, Md., USA) containing 10% fetal bovine serum and 3.6 mM Glutamine. Using Seaplaque GTG agarose (BioWhitaker, Rockland, Me., USA), positive recombinant baculoviruses were plaque-purified and subsequently tested in polyhedrin-specific PCRs for the presence of the L1-genes.

### Example 6

### SDS-PAGE and Immunoblotting

Insect cells were cultured in supplemented Grace's medium (Gibco/BRL, Gaithersburg, Md.) containing 10% fetal bovine serum. Cells were incubated for 2 h in 6 cm diameter Petri dishes at a multiple of infection of 100 infectious recombinant baculoviruses. Seventy-two hours post-infection, cells were collected and suspended in 1 ml RIPA-buffer (1% NP-40, 150 mM NaCl, 1 mM EDTA, 1% DOC, and 0.1% SDS) for 30 min at room temperature (RT). Insoluble fractions were collected by centrifugation in an Eppendorf microcentrifuge at 12,000 rpm for 30 minutes. Proteins were then electrophoretically separated on a 10% SDS-PAGE. Expression of L1 proteins was identified by staining the gel with 0.025% Coomasie brilliant blue R (Sigma, St. Louis, Mo.). For immunoblotting, proteins were separated on a 10% SDS-PAGE and were electrophoretically transferred to a nitrocellulose membrane. L1 -specific monoclonal antibodies were used as primary antibodies and alkaline-phosphatase-tagged goat anti-mouse IgG (H&L) chains were used as secondary antibodies.

### Example 7

### VLP Purification

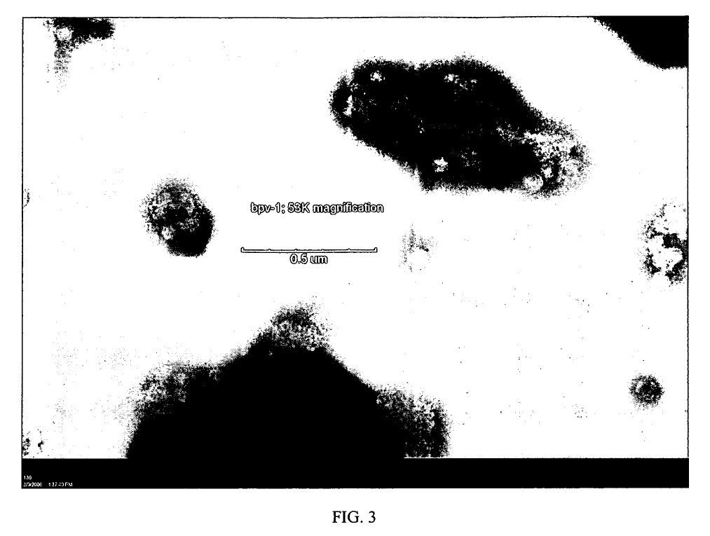

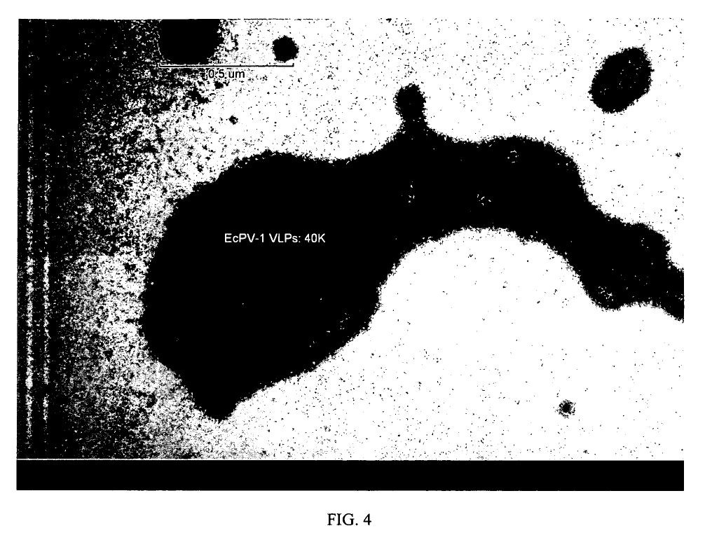

Seventy-two hrs post infection, insect cells were harvested and processed for VLP purification. Briefly, cells were pelleted by centrifugation (170 g, 10 min, 4° C.) and diluted in Dulbecco's Phosphate-Buffered Saline (DPBS), Gibco/BRL (Gaithersburg, Md., USA). After dounce homogenization and sonication, 2×CsCl/DPBS was added with a final CsCl density of 1.33 g/cm³, which was confirmed by measuring the refractive index. After differential ultracentrifugation at 45,000 g for 18 hrs at 4° C., bands of correct density containing the VLPs were collected and dialyzed in dialysis cassettes (Slide-A-Lyzer®, Pierce, Rockford, Ill., USA) at 4° C. against 500× the amount of DPBS buffer for 30 min and with exchanged buffer for another 2 hrs. The final dialysis was then performed for 24-48 hrs in fresh DPBS buffer at 4° C. Expression of the corresponding L1 genes was identified using purified VLPs in sodium dodecyl sulfate polyacrylamide gels (SDS-PAGE) and subsequent immunoblotting. A negative staining with 1.5-2% tungstophosphoric acid (pH 6.8) of the purified VLPs was carried out to confirm by transmission electron microscopy that the self-assembled VLPs had icosahedral symmetry, and that the self-assembled VLPs were of their characteristic size (FIG. 3 and 4) (Philips CM12 Transmission Electron Microscope, University of Louisville, Louisville, Ky., USA).

### Example 8

### ELISA Studies

ELISA microplate (Dynatech Laboratories, Inc., Chantilly, Va.) wells were coated with: 0.1 μg PV VLPs or control-VLPs as intact antigen, or with up to 4 μg VLPs in denaturation buffer (1% SDS, 0.25 mM 2-mercaptoethanol, 15 mM NaCl, 20 mM Tris, pH 7.4) as disrupted antigen as described in Ghim, et al., Biochem Biophys Res Comm. Nov. 19, 2004; 324 (3): 1108-1115, which is incorporated herein by reference. Blocking was performed with PBS containing 5% BSA (bovine serum albumin). The coated wells were incubated with primary antibodies in PBS containing 1% bovine serum albumin (PBSA) and then with the appropriate alkaline-phosphatase-conjugated goat anti-IgG (H&L chains) secondary antibodies at a 1/1000 dilution in PBSA. Incubation steps were performed for 1 h at 37° C., and three to five PBS-washing steps were conducted after each incubation. The adsorption was then measured at 405-410 nm (Spectra MR™, Dynex Technologies, Chantilly, Va., USA) using AP chromogenic substrate (Sigma 104® p-Nitrophenyl Phosphate; Sigma, St. Louis, Mo.), with 1% PBSA used as a negative control.

### Example 9

### Efficacy of Composition in Horses

The effectiveness of a composition including EcPV and BPV VLPs was investigated using seven horses. The average age of the test horses was seven years with an average weight of 1005 pounds. The group included mares, studs, and geldings. The study also enabled a retrospective examination of the persistence of immune response to previous PV exposure.

After an initial prebleed to establish baseline antibody levels, the first vaccination with a 500 μg dose of each immunogen (Immunogen: BPV-1 VLPs and EcPV-1 VLPs purified from insect cells expressing capsid proteins of BPV-1 or EcPV-1; Treatment of Immunogen: fixed with formalin at 1/10,000 dilution) per horse was administered. The seven horses were divided into three groups: 2 animals received EcPV-1 only; three received BPV-1 only; and 2 received the bivalent vaccine of EcPV-1 plus BPV-1. Two weeks later, the animals were bled again and received the second inoculation of vaccine. A third bleed was done two weeks later, and the final bled was done at week 16. A large animal veterinarian examined the vaccinated horses and confirmed there were no clinical adverse reactions.

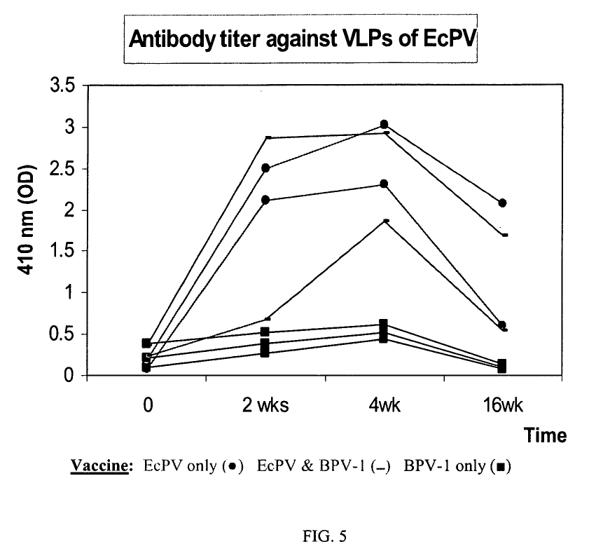

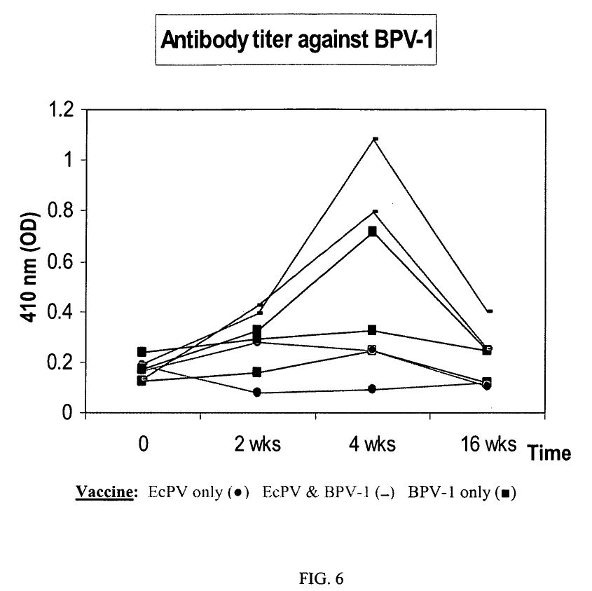

Using an ELISA assay, the collected sera was analyzed for the presence of antibodies to the immunogens. The immune response results appear to distinguish a primary response from a secondary response. With reference to FIG. 5, when the antibody titer against VLPs of EcPV was determined, 3 of the 4 animals that received vaccine with EcPV demonstrated a secondary response while the fourth showed a primary response. The three horses that were inoculated with BPV-1 did not respond to the VLPs of EcPV for detection of antibodies against EcPV, which shows the specificity of the VLPs. With reference to FIG. 6, when the antibody titer against BPV-1 was determined, surprisingly, the horses responded poorly to BPV-1, except when given as a bivalent vaccine.

The primary responses seen in the results of the study described herein indicate that a noninfectious, nonreplicative vaccine, as well as a bivalent vaccine, elicits a primary immune response in animals that have not been previously infected. The secondary responses seen in the results of the study described herein indicate that a noninfectious, nonreplicative VLP vaccine, as well as a bivalent VLP vaccine, elicits a secondary immune response in animals, which had been infected as long as five years previously with no serious side effects.

These results indicate that an immune response against a vaccine booster appears capable of distinguishing a primary antibody response from a secondary response, thus determining whether a subject is still protected by a preceding natural infection or vaccination. This could have clinical implications for determining the length of effectiveness of the immune response in subjects receiving booster immunizations for PV vaccines.

One year after the first vaccination of the seven horses was conducted, all of the horses were given a booster with about 500 μg of: EcPV-1 only (2 animals); BPV-1 only (3 animals); and bivalent vaccine of EcPV-1 plus BPV-1 (2 animals). All horses responded with a brisk secondary immune response.

Throughout this application, various publications are referenced. All such references are incorporated herein by reference, including the references set forth in the following list.
Batzer, et al. Enhanced evolutionary PCR using oligonucleotides with inosine at the 3′-terminus. Nucleic Acids Res. 1991; 19:5081.
Ghim, et al. Equine Papillomavirus type 1: complete nucleotide sequence and characterization of recombinant virus-like particles composed of the EcPV-1 L1 major capsid protein. Biochem, Biophys Res Comm. Nov. 19, 2004; 324 (3): 1108-1115.
Klinman, et al. CpG Motifs Present in Bacterial DNA Rapidly Induce Lymphocytes to Secrete Interleukin 6, Interleukin 12, and Interleukin γ. Proc. Natl. Acad. Sci. USA 1996; 93:2879-2883.
Ohtsuka, et al. An alternative approach to deoxyoligonucleotides as hybridization probes by insertion of deoxyinosine at ambiguous codon positions. J Biol Chem 1985; 260:2605-2608.
Powell M F and Newman M J. Vaccine Design: The Subunit and Adjuvant Approach, Plenum Press, New York, 1995.
Rector, et al. Characterization of a novel close-to-root papillomavirus from a Florida manatee by using multiply primed rolling-circle amplification: Trichechus manatus latirostris papillomavirus type 1. Journal of Virology 2004; 78(22):12698-12702.
Rossolini et al. Use of deoxyinosine-containing primers vs. degenerate primers for polymerase chain reaction based on ambiguous sequence information. Mol Cell Probes 1994; 8(2):91-98.
U.S. Pat. No. 5,057,411 to LANCASTER, et al., issued Oct. 15, 1991, entitled “Type-specific papillomavirus DNA sequences and peptides.”
U.S. Pat. No. 5,874,089 to SCHLEGEL, et al., issued Feb. 23, 1999, entitled “Protecting against canine oral papillomavirus.”
U.S. Pat. No. 6,153,201 to ROSE, et al., issued Nov. 28, 2000, entitled “Oral immunization with papillomavirus virus-like particles.”
U.S. Pat. No. 6,165,471 to GARCEA, et al., issued Dec. 26, 2000, entitled “Homogeneous human papillomavirus capsomere containing compositions, methods for manufacture, and use thereof as diagnostic, prophylactic or therapeutic agents.”
U.S. Pat. No. 6,485,728 to SCHLEGEL, et al., issued Nov. 26, 2002, entitled “Formalin-Inactivated human papillomavirus L1 protein vaccine.”
U.S. Pat. No. 6,887,478 to SCHLEGEL, et al., issued May 3, 2005, entitled “Formalin-treated human papillomavirus L1 protein vaccine.”
U.S. Pat. No. 6,908,615 to HOFMANN, et al., issued Jun. 21, 2005, entitled “DNA encoding human papilloma virus type 18.”
U.S. Pat. No. 7,001,995 to NEEPER, et al., issued Feb. 21, 2006, entitled “Synthetic human papillomavirus genes.”
United States Patent Application Publication No. 2002/0197264 of Schlegel, et al.
United States Patent Application Publication No. 2004/0086527 of Schlegel, et al.
United States Patent Application Publication No. 2005/0026257 of Gissmann, et al.
United States Patent Application Publication No. 2005/0282263 of McCormick, et al.
United States Patent Application Publication No. 2006/0029612 of Palmer, et al.
International Patent Application Publication No. WO98/16247 of Carson, et al.

What is claimed is:

1. A composition for conferring protection against Equus caballus papillomavirus (EcPV) and Bovine papillomavirus (BPV) infection in a subject susceptible to infection, comprising:
a virus-like particle assembled from an isolated EcPV L1 protein; and
a virus-like particle assembled from an isolated BPV L1 protein.

2. The composition of claim 1, wherein said BPV is selected from the group consisting of BPV-1 and BPV-2.

3. The composition of claim 2, wherein said BPV is BPV-1 and said EcPV is EcPV-1.

4. The composition of claim 2, wherein said BPV is BPV-2 and said EcPV is EcPV-1.

5. The composition of claim 1, comprising:
a virus-like particle assembled from an isolated EcPV-1 L1 protein;
a virus-like particle assembled from an isolated BPV-1 L1 protein; and
a virus-like particle assembled from an isolated BPV-2 L1 protein.

6. The composition of claim 1,
(a) wherein the EcPV L1 protein is a functional polypeptide comprising a polypeptide selected from:
a sequence of SEQ ID NO: 1;
a fragment of SEQ ID NO: 1; and
a polypeptide comprising the sequence of SEQ ID NO: 1 with up to 2 conservative amino acids substitutions; and
(b) wherein the BPV L1 protein is a functional polypeptide comprising a polypeptide selected from:
(i) the sequence of SEQ ID NO: 3;
a fragment of SEQ ID NO: 3; or
a polypeptide comprising the sequence of SEQ ID NO: 3 with up to 2 conservative amino acids substitutions; and
(ii) the sequence of SEQ ID NO: 5;
a fragment of SEQ ID NO: 5; or
a polypeptide comprising the sequence of SEQ ID NO: 5 with up to 2 conservative amino acids substitutions.

7. The composition of claim 6, wherein the EcPV L1 protein is a functional polypeptide comprising a fragment of SEQ ID NO: 1 and, wherein the fragment comprises about amino acid 1 to about amino acid 480 of SEQ ID NO: 1.

8. The composition of claim 6, wherein the EcPV L1 protein is a functional polypeptide comprising a fragment of SEQ ID NO: 1 and, wherein the fragment comprises about amino acid 1 to about amino acid 479 of SEQ ID NO: 1.

9. The composition of claim 6, wherein the BPV L1 protein is a functional polypeptide comprising a fragment of SEQ ID NO: 3 and, wherein the fragment comprises about amino acid 1 to about amino acid 470 of SEQ ID NO: 3.

10. The composition of claim 6, wherein the BPV L1 protein is a functional polypeptide comprising a fragment of SEQ ID NO: 3 and, wherein the fragment comprises about amino acid 1 to about amino acid 469 of SEQ ID NO: 3.

11. The composition of claim 6, wherein the BPV L1 protein is a functional polypeptide comprising a fragment of SEQ ID NO: 5 and, wherein the fragment comprises about amino acid 1 to about amino acid 472 of SEQ ID NO: 5.

12. The composition of claim 6, wherein the BPV L1 protein is a functional polypeptide comprising a fragment of SEQ ID NO: 5 and, wherein the fragment comprises about amino acid 1 to about amino acid 471 of SEQ ID NO: 5.

13. A composition for conferring protection against Equus caballus papillomavirus (EcPV) and Bovine papillomavirus (BPV) infection in a subject susceptible to infection, comprising:
(a) a virus like particle assembled from an isolated polypeptide selected from:
(i) a functional polypeptide being encoded by
a nucleic acid molecule comprising
the sequence of SEQ ID NO: 2;
a degenerate variant of SEQ ID NO: 2; or
a fragment of SEQ ID NO: 2; and
(ii) a functional polypeptide comprising
the sequence of SEQ ID NO: 1;
a fragment of SEQ ID NO: 1; or
a polypeptide comprising the sequence of SEQ ID NO: 1 with up to 2 conservative amino acid substitutions; and
(b) a virus like particle assembled from an isolated polypeptide selected from:
(i) a functional polypeptide being encoded by
a nucleic acid molecule comprising
the sequence of SEQ ID NO: 4;
a degenerate variant of SEQ ID NO: 4; or
a fragment of SEQ ID NO: 4;
(ii) a functional polypeptide comprising
the sequence of SEQ ID NO: 3;
a fragment of SEQ ID NO: 3; or
a polypeptide comprising the sequence of SEQ ID NO: 3 with up to 2 conservative amino acid substitutions;
(iii) a functional polypeptide being encoded by
a nucleic acid molecule comprising
the sequence of SEQ ID NO: 6;
a degenerate variant of SEQ ID NO: 6; or
a fragment of SEQ ID NO: 6; and
(iv) a functional polypeptide comprising
the sequence of SEQ ID NO: 5;
a fragment of SEQ ID NO: 5; or
a polypeptide comprising the sequence of SEQ ID NO: 5 with up to 2 conservative amino acid substitutions.

14. A method of protecting a subject against Equus caballus papillomavirus (EcPV) and Bovine papillomavirus (BPV) infection by administering an effective amount of the composition of claim 13.

15. A method of protecting a subject against Equus caballus papillomavirus (EcPV) and Bovine papillomavirus (BPV) infection by administering an effective amount of the composition of claim 1.

16. The method of claim 15, wherein said administered composition comprises a virus-like particle assembled from an isolated EcPV-1 L1 protein; and a virus-like particle assembled from an isolated BPV L1 protein, wherein the BPV is selected from the group consisting of BPV-1 and BPV-2.

17. The method of claim 16, wherein said administered composition comprises a virus-like particle assembled from an isolated BPV-1 L1 protein, and a virus-like particle assembled from an isolated BPV-2 L1 protein.

18. A method of diagnosing Equus caballus papillomavirus (EcPV) and/or Bovine papillomavirus (BPV) infection in a subject, comprising:
providing a virus-like particle assembled from an isolated PV L1 protein selected from:
EcPV L1 protein; and
BPV L1 protein;
contacting the virus-like particle with serum obtained from the subject; and
identifying the subject as having an infection if an antibody capable of binding the virus-like particle is detected in the serum.

19. The method of claim 18, further comprising:
providing a virus-like particle assembled from an isolated EcPV L1 protein and a virus-like particle assembled from an isolated BPV L1 protein;
identifying the subject as having an EcPV infection if an antibody capable of binding the EcPV virus-like particle is detected in the serum; and
identifying the subject as having a BPV infection if an antibody capable of binding the BPV virus-like particle is detected in the serum.

20. The method of claim 19, wherein the binding is detected using an antibody capable of binding the EcPV antibody, and an antibody capable of binding the BPV antibody.
